(19) United States
(12) Patent Application Publication
(10) Pub. No.: US 2022/0309215 A1
(43) Pub. Date: Sep. 29, 2022

(54) METHODS AND SYSTEMS FOR DEVELOPING MIXING PROTOCOLS

(71) Applicant: Regeneron Pharmaceuticals, Inc., Tarrytown, NY (US)

(72) Inventors: Ross KENYON, Saratoga Springs, NY (US); Jordan BYRD, East Greenbush, NY (US); Matthew OREMLAND, Clifton Park, NY (US)

(73) Assignee: Regeneron Pharmaceuticals, Inc., Tarrytown, NY (US)

(21) Appl. No.: 17/656,617

(22) Filed: Mar. 25, 2022

Related U.S. Application Data
(60) Provisional application No. 63/298,880, filed on Jan. 12, 2022.
(60) Provisional application No. 63/166,504, filed on Mar. 26, 2021.

(51) Int. Cl. G06F 30/28

(52) U.S. Cl. CPC G06F 30/28

(57) ABSTRACT

A method of developing a predictive model may include identifying mixing protocol parameters for the predictive model, identifying an evaluation criterion for the predictive model, selecting test values for the mixing protocol parameters, identifying a computational fluid dynamics (CFD) simulation required to be performed in order to generate the evaluation criteria, conducting the CFD simulation for each combination of test values, thereby generating evaluation criteria corresponding to each combination of test values, generating a domain of potential predictive models relating the mixing protocol parameters to the evaluation criterion, identifying a pool of candidate predictive models from the domain of potential predictive models, and ranking the pool of candidate predictive models.

### CROSS-REFERENCE TO RELATED APPLICATIONS

This application claims priority to U.S. Provisional Patent Application No. 63/166,504, filed on March 26, 2021, and U.S. Provisional Patent Application No. 63/298,880, filed on Jan. 12, 2022, both of which are hereby incorporated by reference in their entirety.

### FIELD OF DISCLOSURE

The present disclosure relates to systems and methods for developing and implementing mixing protocols. Some aspects of the present disclosure relate to systems and methods for high-throughput evaluation of mixing protocols related to the biological production of therapeutics.

### INTRODUCTION

Biopharmaceutical products (e.g., antibodies, fusion proteins, adeno-associated viruses (AAVs), proteins, tissues, cells, polypeptides, or other therapeutic products of biological origin) are increasingly being used in the treatment and prevention of infectious diseases, genetic diseases, autoimmune diseases, and other ailments. Production of the biopharmaceutical products requires precise and consistent conditions. In order to ensure that solutions including biopharmaceutical products are consistent, mixing protocols may be employed throughout the manufacturing process. The mixing protocols may assist in maintaining suitable distribution of solution components (e.g., biopharmaceutical products, cell waste, host protein, extracellular nutrients, other molecules) within the various solutions involved in the production of biopharmaceutical products.

Mixing protocols may include parameters for shape and size of a mixing vessel, direction and rate of fluid flow within the solution, and physiochemical properties of the solution. Mixing protocols may be developed for each type of biopharmaceutical product, mixing vessel geometry, media composition, and host cell. Modifications to the biopharmaceutical product, mixing vessel geometry, media composition, or host cell may require redevelopment of the mixing protocol. Conventional methods of developing a mixing protocol are time and labor intensive, and may result in an inferior mixing protocol.

### SUMMARY

Embodiments of the present disclosure may be directed to a method of developing a predictive model. The method may include identifying mixing protocol parameters for the predictive model, identifying an evaluation criterion for the predictive model, and/or selecting test values for the mixing protocol parameters. The method may further include identifying a computation fluid dynamics (CFD) simulation required to be performed in order to generate the evaluation criterion. The method may also include conducting the CFD simulation for each combination of test values, thereby generating evaluation criteria corresponding to each combination of test values. The method may further include generating a domain of potential predictive models relating the mixing protocol parameters to the evaluation criterion, identifying a pool of candidate predictive models from the domain of potential predictive models, and/or ranking the pool of candidate predictive models.

In some embodiments of the present disclosure, the mixing protocol parameters may include two or more of impeller speed, batch size, solution viscosity, solution density, mixing vessel size, and mixing vessel geometry. The evaluation criteria may include two or more of: flow pattern, fluid velocity distribution, fluid flow vector field, fluid flow streamlines, steady state blend time, transient blend time, residence time distribution, contour shear strain rate, average shear strain rate, exposure analysis, and power consumption. The identified CFD simulation may include a steady flow analysis, a transient flow analysis, a blend time analysis, and/or an exposure analysis. In some embodiments, the method of developing a mixing predictive model may further include, after generating a domain of potential predictive models, and prior to identifying a pool of candidate predictive models, calculating a variance inflation factor for each potential predictive model in the domain of potential predictive models, and removing potential predictive models from the domain of potential predictive models that have a variance inflation factor greater than or equal to a collinearity threshold, thereby generating a subset of potential predictive models. The pool of candidate predictive models may include a univariate model from the subset that has a R² value higher than all other univariate models in the subset, and a bivariate model from the subset that has a R² value higher than all other bivariate models in the subset. Ranking the pool of candidate predictive models may include ranking the pool of candidate predictive models based on number of terms, ranking the pool of candidate predictive models based on R² value, or both. In some embodiments of the present disclosure, the test values are first test values, and the method of developing a predictive model further comprises generating an estimated value of the evaluation criteria corresponding to a combination of second test values, using a candidate predictive model from the pool of candidate predictive models. In addition, the method may further comprise conducting the CFD simulation for the combination of second test values to generate an evaluation criterion corresponding to the combination of second test values, and comparing the evaluation criterion corresponding to the combination of second test values with the estimated value of the evaluation criterion corresponding to the combination of second test values.

Further embodiments of the present disclosure may include a method of developing predictive models. The method may include identifying first, second, and third mixing protocol parameters for the predictive models, identifying first and second evaluation criteria for the predictive models, selecting first test values for the first mixing protocol parameter, selecting second test values for the second mixing protocol parameter, and/or selecting third test values for the third mixing protocol parameter. The method may further include identifying a first computational fluid dynamics (CFD) simulation required to be performed in order to generate the first evaluation criterion, identifying a second CFD simulation required to be performed in order to generate the second evaluation criterion; generating a first evaluation criterion corresponding to each combination of first test values, second values, and third test values, by performing the first CFD simulation for each combination of first test values, second values, and third test values; and/or generating a second evaluation criterion corresponding to each combination of first test values, second values, and third test values, by performing the second CFD simulation for each combination of first test values, second values, and third test values. The method may further include generating a first domain of first predictive models relating the first, second, and third mixing protocol parameters to the first evaluation criterion and/or generating a second domain of second predictive models relating the first, second, and third mixing protocol parameters to the second evaluation criterion.

In some embodiments of the present disclosure, a method of developing predictive models may further include calculating a variance inflation factor for each first predictive model and each second predictive model, removing first predictive models from the first domain of first predictive models that have a variance inflation factor greater than or equal to three, thereby generating a first subset of first predictive models, removing second predictive models from the second domain of second predictive models that have a variance inflation factor greater than or equal to three, thereby generating a first subset of first predictive models, identifying a first pool of candidate first predictive models comprising a univariate model from the first subset that has a R2 value higher than all other univariate models in the first subset, a bivariate model from the first subset that has a R2 value higher than all other bivariate models in the first subset, and a trivariate model from the first subset that has a R2 value higher than all other trivariate models in the first subset, identifying a second pool of candidate second predictive models comprising a univariate model from the second subset that has a R2 value higher than all other univariate models in the second subset, a bivariate model from the second subset that has a R2 value higher than all other bivariate models in the second subset, and a trivariate model from the second subset that has a R2 value higher than all other trivariate models in the second subset, selecting fourth test values for the first mixing protocol parameter, selecting fifth test values for the second mixing protocol parameter, selecting sixth test values for the third mixing protocol parameter, generating an estimated first evaluation criterion corresponding to each combination of fourth test values, fifth test values, and sixth test values, using each candidate first predictive model of the first pool of candidate first predictive models, generating a first evaluation criterion corresponding to each combination of fourth test values, fifth test values, and sixth test values, by performing the first CFD simulation for each combination of fourth test values, fifth test values, and sixth test values, comparing the estimated first evaluation criterions generated by each candidate first predictive model of the first pool of candidate first predictive models to the first evaluation criterions corresponding to each combination of fourth test values, fifth test values, and sixth test values, generating an estimated second evaluation criterion corresponding to each combination of fourth test values, fifth test values, and sixth test values, using each candidate second predictive model of the second pool of candidate second predictive models, generating a second evaluation criterion corresponding to each combination of fourth test values, fifth test values, and sixth test values, by performing the second CFD simulation for each combination of fourth test values, fifth test values, and sixth test values, and comparing the estimated second evaluation criterions generated by each candidate second predictive model of the second pool of candidate second predictive models to the second evaluation criterions corresponding to each combination of fourth test values, fifth test values, and sixth test values, selecting a first predictive model from the first pool of candidate first predictive models, based on the comparison the estimated first evaluation criterions to the first evaluation criterions corresponding to each combination of fourth test values, fifth test values, and sixth test values, selecting a second predictive model from the second pool of candidate second predictive models, based on the comparison the estimated first evaluation criterions to the first evaluation criterions corresponding to each combination of fourth test values, fifth test values, and sixth test values, using the first predictive model, determining a first evaluation criterion corresponding to a mixing protocol, and using the second predictive model, determining a second evaluation criterion corresponding to the mixing protocol.

Further embodiments of the present disclosure may include a method of modeling shear strain associated with a mixing protocol. The method may include identifying mixing protocol parameters for a predictive model, selecting test values for the mixing protocol parameters, conducting a computational fluid dynamics exposure analysis for each of combination of test values, thereby generating a shear strain corresponding to each combination of test values, identifying a pool of candidate predictive models, ranking the pool of candidate predictive models, selecting a predictive model from the pool of candidate predictive models, and using the predictive model, evaluating cumulative shear strain of the mixing protocol at a plurality of time intervals to generate shear strain histogram data.

In some embodiments of the present disclosure, a method of modeling shear strain associated with a mixing protocol includes wherein the mixing protocol parameters include two or more of: impeller speed, batch size, solution viscosity, solution density, mixing vessel size, and mixing vessel geometry. Ranking the pool of candidate predictive models includes ranking the pool of candidate predictive models based on number of terms, ranking the pool of candidate predictive models based on R² value, or both, and selecting a predictive model from the pool of candidate predictive models includes selecting the model with the highest R² value The mixing protocol may be a mixing protocol associated with biopharmaceutical products in a bioreactor. The method may further comprise using the shear strain histogram data to assess the risk of visible or sub-visible particle formation.

### BRIEF DESCRIPTION OF THE DRAWINGS

The accompanying drawings, which are incorporated in and constitute a part of this specification, illustrate various exemplary embodiments, and together with the description, serve to explain the principles of the disclosed embodiments. Any features of an embodiment or example described herein (e.g., composition, formulation, method, etc.) may be combined with any other embodiment or example, and all such combinations are encompassed by the present disclosure. Moreover, the described systems and methods are neither limited to any single aspect nor embodiment thereof, nor to any combinations or permutations of such aspects and embodiments. For the sake of brevity, certain permutations and combinations are not discussed and/or illustrated separately herein.

FIG. 1 depicts a strain histogram, according to aspects of the present disclosure;

FIG. 2 depicts, in flow-chart form, an exemplary method for developing a predictive model for evaluating mixing protocols, according to aspects of the present disclosure;

FIGS. 3A and 3B are graphical representations of mixing vessels, according to aspects of the present disclosure;

FIG. 4A is a visual depiction of a fluid flow vector field, according to aspects of the present disclosure;

FIG. 4B is a visual depiction of fluid flow streamlines, according to aspects of the present disclosure;

FIG. 4C is a visual depiction of contour shear strain rate, according to aspects of the present disclosure;

FIG. 5 depicts, in flow-chart form, an exemplary method for building potential predictive models, according to aspects of the present disclosure;

FIG. 6 depicts a plot of blend time determined by CFD analysis versus blend time determined by predictive model, according to aspects of the present disclosure;

FIG. 7 depicts a plot of strain rate determined by CFD analysis versus strain rate determined by predictive model, according to aspects of the present disclosure;

FIG. 8 depicts a strain rate histogram generated by plotting a predictive model, according to aspects of the present disclosure;

FIGS. 9A-9C are a visual depiction of a theorized mechanism of aggregate formation, according to aspects of the present disclosure;

FIG. 10A is a visual depiction of vertical velocity contours, according to aspects of the present disclosure;

FIG. 10B is a visual depiction of volume-average velocity, according to aspects of the present disclosure; and

FIG. 11 depicts a plot of vertical velocity as a function of tank radius, according to aspects of the present disclosure.

### DETAILED DESCRIPTION

Unless defined otherwise, all technical and scientific terms used herein have the same meaning as is commonly understood by one of ordinary skill in the art to which this disclosure belongs. Although any suitable methods and materials (e.g., similar or equivalent to those described herein) can be used in the practice or testing of the present disclosure, particular example methods are now described. All publications mentioned are hereby incorporated by reference.

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” For the terms “for example” and “such as,” and grammatical equivalences thereof, the phrase “and without limitation” is understood to follow unless explicitly stated otherwise.

As used herein, the term “about” is meant to account for variations due to experimental error. When applied to numeric values, the term “about” may indicate a variation of +/−5% from the disclosed numeric value, unless a different variation is specified. As used herein, the singular forms “a,” “an,” and “the” include plural referents unless the context clearly dictates otherwise. Further, all ranges are understood to be inclusive of endpoints, e.g., from 1 centimeter (cm) to 5 cm would include lengths of 1 cm, 5 cm, and all distances between 1 cm and 5 cm.

It should be noted that all numeric values disclosed or claimed herein (including all disclosed values, limits, and ranges) may have a variation of +/−5% from the disclosed numeric value unless a different variation is specified.

The term “polypeptide” as used herein refers to any amino acid polymer having more than about 20 amino acids covalently linked via amide bonds. Proteins contain one or more amino acid polymer chains (e.g., polypeptides). Thus, a polypeptide may be a protein, and a protein may contain multiple polypeptides to form a single functioning biomolecule.

Post-translational modifications may modify or alter the structure of a polypeptide. For example, disulfide bridges (e.g., S—S bonds between cysteine residues) may be formed post-translationally in some proteins. Some disulfide bridges are essential to proper structure, function, and interaction of polypeptides, immunoglobulins, proteins, co-factors, substrates, and the like. In addition to disulfide bond formation, proteins may be subject to other post-translational modifications, such as lipidation (e.g., myristoylation, palmitoylation, farnesoylation, geranylgeranylation, and glycosylphosphatidylinositol (GPI) anchor formation), alkylation (e.g., methylation), acylation, amidation, glycosylation (e.g., addition of glycosyl groups at arginine, asparagine, cysteine, hydroxylysine, serine, threonine, tyrosine, and/or tryptophan), and phosphorylation (i.e., the addition of a phosphate group to serine, threonine, tyrosine, and/or histidine). Post-translational modifications may affect the hydrophobicity, electrostatic surface properties, or other properties which determine the surface-to-surface interactions participated in by the polypeptide.

As used herein, the term “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, antibody-like molecules, nanobodies, recombinant antibody chimeras, cytokines, chemokines, peptide hormones, and the like. A protein of interest (POI) may include any polypeptide or protein that is desired to be isolated, purified, or otherwise prepared. POIs may include polypeptides produced by a cell, including antibodies.

The term “antibody,” as used herein, includes immunoglobulins comprised of four polypeptide chains: two heavy (H) chains and two light (L) chains inter-connected by disulfide bonds. Typically, antibodies have a molecular weight of over 100 kDa, such as between 130 kDa and 200 kDa, such as about 140 kDa, 145 kDa, 150 kDa, 155 kDa, or 160 kDa. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3.

A class of immunoglobulins called Immunoglobulin G (IgG), for example, is common in human serum and comprises four polypeptide chains—two light chains and two heavy chains. Each light chain is linked to one heavy chain via a cystine disulfide bond, and the two heavy chains are bound to each other via two cystine disulfide bonds. Other classes of human immunoglobulins include IgA, IgM, IgD, and IgE. In the case of IgG, four subclasses exist: IgG 1, IgG 2, IgG 3, and IgG 4. Each subclass differs in their constant regions, and as a result, may have different effector functions. In some embodiments described herein, a POI may comprise a target polypeptide including IgG. In at least one embodiment, the target polypeptide comprises IgG 4.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Target molecules (such as target polypeptides/antibodies) may be produced using recombinant cell-based production systems, such as the insect bacculovirus system, yeast systems (e.g., Pichia sp.), or mammalian systems (e.g., CHO cells and CHO derivatives like CHO-K1 cells). The term “cell” includes any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, bacculovirus-infected insect cells, Trichoplusiani, etc.), non-human animal cells, human cells, or cell fusions such as, for example, hybridomas or quadromas. In some embodiments a cell may be a human, monkey, ape, hamster, rat, or mouse cell. In some embodiments, a cell may be eukaryotic and may be selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, a cell may comprise one or more viral genes, e.g. a retinal cell that expresses a viral gene (e.g., a PER.C6™ cell).

The term “target molecule” may be used herein to refer to target polypeptides (e.g., antibodies, antibody fragments, or other proteins or protein fragments), or to other molecules intended to be produced, isolated, purified, and/or included in drug products (e.g., adeno-associated viruses (AAVs) or other molecules for therapeutic use). While methods according to the present disclosure may refer to target polypeptides, they may be as applicable to other target molecules. AAVs, for example, may be prepared according to suitable methods (e.g., depth filtration, affinity chromatography, and the like), and mixtures including AAVs may be subjected to methods according to the present disclosure. Before or after following one or more methods of the present disclosure, mixtures including AAVs may be subjected to additional procedures (e.g., to the removal of “empty cassettes” or AAVs that do not contain a target sequence).

In some embodiments, the target molecule is an antibody, a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody. In one embodiment, the antibody is a chimeric IgG2/IgG4 antibody. In one embodiment, the antibody is a chimeric IgG2/IgG1 antibody. In one embodiment, the antibody is a chimeric IgG2/IgG1/IgG4 antibody.

In some embodiments, a target molecule (e.g., an antibody) is selected from a group consisting of an anti-Programmed Cell Death 1 antibody (e.g., an anti-PD1 antibody as described in U.S. Pat. Appin. Pub. No. US2015/0203579A1), an anti-Programmed Cell Death Ligand-1 (e.g. an anti-PD-L1 antibody as described in in U.S. Pat. Appin. Pub. No. US2015/0203580A1), an anti-D114 antibody, an anti-Angiopoetin-2 antibody (e.g., an anti-ANG2 antibody as described in U.S. Pat. No. 9,402,898), an anti-Angiopoetin-Like 3 antibody (e.g. an anti-AngPt13 antibody as described in U.S. Pat. No. 9,018,356), an anti-platelet derived growth factor receptor antibody (e.g. an anti-PDGFR antibody as described in U.S. Pat. No. 9,265,827), an anti-Prolactin Receptor antibody (e.g., anti-PRLR antibody as described in U.S. Pat. No. 9,302,015), an anti-Complement 5 antibody (e.g., an anti-05 antibody as described in U.S. Pat. Appin. Pub. No US2015/0313194A1), an anti-TNF antibody, an anti-epidermal growth factor receptor antibody (e.g., an anti-EGFR antibody as described in U.S. Pat. No. 9,132,192 or an anti-EGFRvIII antibody as described in U.S. Pat. Appin. Pub. No. US2015/0259423A1), an anti-Proprotein Convertase Subtilisin Kexin-9 antibody (e.g., an anti-PCSK9 antibody as described in U.S. Pat. No. 8,062,640 or U.S. Pat. Appin. Pub. No. US2014/0044730A1), an anti-Growth And Differentiation Factor-8 antibody (e.g., an anti-GDF8 antibody, also known as anti-myostatin antibody, as described in U.S. Pat Nos. 8,871,209 or 9,260,515), an anti-Glucagon Receptor (e.g., anti-GCGR antibody as described in U.S. Pat. Appin. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. Appin. Pub. No. US2014/0271681A1 or U.S. Pat Nos. 8,735,095 or 8,945,559), an anti-interleukin 6 receptor antibody (e.g., an anti-IL6R antibody as described in U.S. Pat. Nos. 7,582,298, 8,043,617 or 9,173,880), an anti-interleukin 33 (e.g., anti-IL33 antibody as described in U.S. Pat. Appin. Pub. Nos. US2014/0271658A1 or US2014/0271642A1), an anti-Respiratory syncytial virus antibody (e.g., anti-RSV antibody as described in U.S. Pat. Appin. Pub. No. US2014/0271653A1), an anti-Cluster of differentiation 3 (e.g., an anti-CD3 antibody, as described in U.S. Pat. Appin. Pub. Nos. US2014/0088295A1 and US20150266966A1, and in U.S. Application No. 62/222,605), an anti-Cluster of differentiation 20 (e.g., an anti-CD20 antibody as described in U.S. Pat. Appin. Pub. Nos. US2014/0088295A1 and US20150266966A1, and in U.S. Pat. No. 7,879,984), an anti-Cluster of Differentiation-48 (e.g., anti-CD48 antibody as described in U.S. Pat. No. 9,228,014), an anti-Fel d1 antibody (e.g., as described in U.S. Pat. No. 9,079,948), an anti-Middle East Respiratory Syndrome virus (e.g., an anti-MERS antibody), an anti-Ebola virus antibody (e.g., Regeneron's REGN-EB3), an anti-CD19 antibody, an anti-CD28 antibody, an anti-IL1 antibody, an anti-IL2 antibody, an anti-IL3 antibody, an anti-IL4 antibody, an anti-IL5 antibody, an anti-IL6 antibody, an anti-IL7 antibody, an anti-Erb3 antibody, an anti-Zika virus antibody, an anti-Lymphocyte Activation Gene 3 (e.g., anti-LAG3 antibody or anti-CD223 antibody) and an anti-Activin A antibody. Each U.S. patent and U.S. patent publication mentioned in this paragraph is incorporated by reference in its entirety.

In some embodiments, a target molecule (e.g., a bispecific antibody) is selected from the group consisting of an anti-CD3×anti-CD20 bispecific antibody, an anti-CD3×anti-Mucin 16 bispecific antibody, and an anti-CD3×anti-Prostate-specific membrane antigen bispecific antibody. In some embodiments, the target molecule is selected from the group consisting of alirocumab, sarilumab, fasinumab, nesvacumab, dupilumab, trevogrumab, evinacumab, and rinucumab.

In some embodiments, the target molecule is a recombinant protein that contains an Fc moiety and another domain, (e.g., an Fc-fusion protein). In some embodiments, an Fc-fusion protein is a receptor Fc-fusion protein, which contains one or more extracellular domain(s) of a receptor coupled to an Fc moiety. In some embodiments, the Fc moiety comprises a hinge region followed by a CH2 and CH3 domain of an IgG. In some embodiments, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-1RAcP ligand binding region fused to the Il-1R1 extracellular region fused to Fc of hIgG1; see U.S. Pat. No. 6,927,004, which is incorporated by reference in its entirety), or a VEGF trap (e.g., aflibercept or ziv-aflibercept, which contains the Ig domain 2 of the VEGF receptor Fla fused to the Ig domain 3 of the VEGF receptor Flk1 fused to Fc of hIgG1; see U.S. Pat. Nos. 7,087,411 and 7,279,159, both of which are incorporated by reference in their entireties). In other embodiments, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

The terms “culture medium” or “medium” refer to a nutrient solution used for growing cells that typically provides the necessary nutrients to enhance growth of the cells, such as a carbohydrate energy source, essential amino acids, trace elements, vitamins, etc. Culture media may contain extracts, e.g., serum or peptones (hydrolysates), which supply raw materials that support cell growth. In some embodiments, instead of animal- derived extracts, media may contain yeast-derived or soy extracts. Chemically defined medium refers to a culture medium in which all of the chemical components are known. Chemically defined medium may be entirely free of animal-derived components, such as serum- or animal-derived peptones. Medium may also be protein-free. “Fresh media” may refer to media that has not yet been introduced into cell culture and/or has not yet been utilized by cells of a cell culture. Fresh media may include generally high nutrient levels and little-to-no waste products. “Spent media” may refer to media that has been used by cells in cell culture, and may generally include lower nutrient levels and higher water levels, compared to fresh media.

Generally, mixing protocols may be incorporated into several stages of the manufacture of biopharmaceutical products. For example, during the culture of host cells or the harvest of a biopharmaceutical product, a mixing protocol may be utilized to ensure suitable distribution of generated biopharmaceutical products, cells, nutrients, waste, and other components of the culture media. The mixing protocol may be employed with a vessel configured to execute a mixing protocol, also referred to as a mixing vessel. In some embodiments, a bioreactor may be used as the mixing vessel. In other embodiments, culture fluid may be transferred from a bioreactor to a different type of mixing vessel prior to execution of a mixing protocol.

After the harvest of a biopharmaceutical product (e.g., a protein of interest), the harvested product may be kept in solution. The solution including the biopharmaceutical product may undergo one or more chromatography, filtration (e.g., ultrafiltration, diafiltration, or a combination thereof), purification (e.g., viral inactivation) steps to increase the purity and effectiveness of the biopharmaceutical product. At all stages, a mixing protocol may be employed to homogenize the solution and/or ensure suitable distribution of solution components. In addition to the applications discussed above, mixing protocols may be employed to combine and/or dilute individual biotainers, batches, or lots.

Additionally, mixing protocols may be applied to solutions not including the protein of interest. For example, the aforementioned steps of biopharmaceutical product manufacture require the use of buffers, media, and other solutions. The preparation of buffers, media, and other solutions may include the use of one or more mixing protocols.

Specific properties of biopharmaceutical products or the manufacturing process thereof that are dependent on the mixing protocol may be monitored to assess the impact of parameters of the mixing protocol on the resulting biopharmaceutical products. For example, the flow pattern, fluid velocity distribution, fluid flow vector field, fluid flow streamlines, blend time (e.g., steady state blend time or transient blend time), residence time distribution, contour shear strain rate, average shear strain rate, exposure analysis and/or power consumption associated with a mixing protocol may be used to assess the utility and/or efficacy of a mixing protocol.

A mixing protocol may include operation parameters for a mixing vessel such as, for example, size of mixing vessel, impeller speed, load size as a percentage of total capacity, viscosity of solution, and/or other operation parameters that describe the requirements of the mixing protocol. In some embodiments, a mixing protocol is complete when the solution (including, e.g., media, cells, protein(s) of interest, and/or other molecules) is sufficiently homogenized. The duration of a mixing protocol, i.e., the time it takes for the solution to reach sufficient homogeneity, is referred to as the blend time. The extent to which a solution has been mixed may be quantified by a mixing index. The mixing index may be defined as the ratio of the standard deviation of concentration (e.g., of a protein of interest or other molecule) to a final concentration. Blend time may be quantified as the amount of time necessary under a given mixing protocol to reach a mixing index of approximately 5%.

In the conventional development of a mixing protocol, physiochemical properties of the protein of interest and the media containing the protein of interest are considered as potential mixing protocols are generated. The potential mixing protocols are tested via surrogate mixing studies to map operating ranges and collect blend time data. Based on the blend time data collected from various points of the operating ranges, one or more candidate mixing protocols may be determined. The candidate mixing protocols may be further tested with shear stress and overmixing studies. The shear stress and overmixing studies may generate product quality data that may be used to evaluate the candidate mixing protocols.

The shear and overmixing studies must be conducted after the blend time data is generated, because shear stress and overmixing are dependent on the blend time. If product quality data provided by the shear stress and overmixing studies indicates that the mixing protocol is unsuitable, the development of the mixing protocol must be restarted to generate potential mixing protocols. Additionally, surrogate mixing studies must be performed for the new potential mixing protocols in order to generate blend time data that can be used for further shear stress and overmixing studies.

This conventional development flow of a mixing protocol is limited because surrogate mixing studies must be performed to evaluate mixing protocols that may eventually result in unfavorable product quality data. The requirement of the conventional development flow to run multiple experiments in order to determine whether a potential mixing protocol should be investigated results in a time and labor intensive development of mixing protocols. Further, events related to an implemented mixing protocol that affect the quality of the resulting biopharmaceutical product, such as air-liquid interfacial stress, air entrainment, and risk of visible or sub-visible particle formation are not addressed in the conventional development flow.

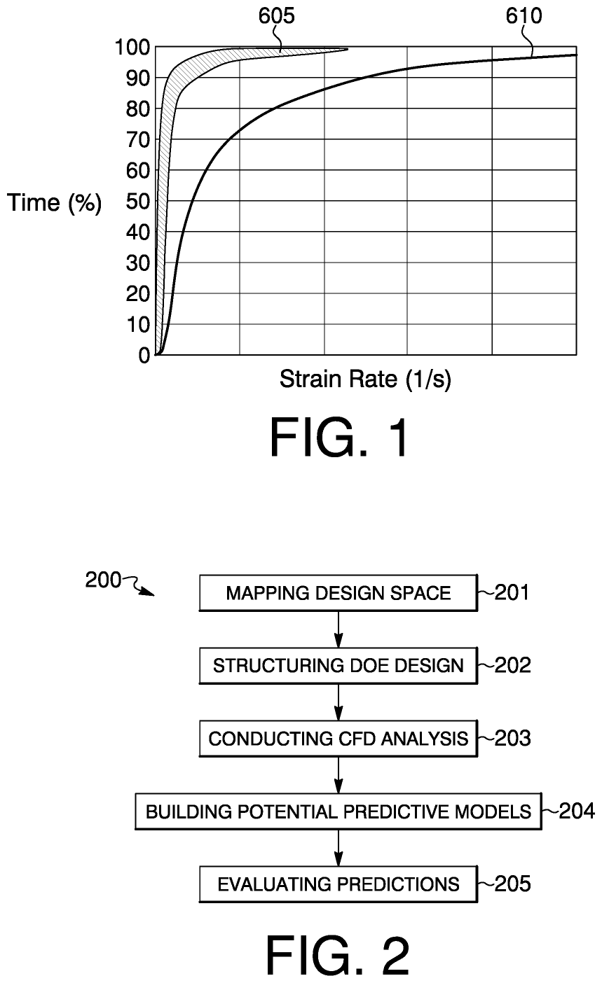

In addition to the failure of conventional mixing protocol development flows to address all factors of a mixing protocol that can lead to adverse consequences for the resulting biopharmaceutical product, the scaled studies result in excessively high shear stress. FIG. 1 shows strain histograms that represent how the scaled shear stress studies associated with conventional mixing protocol development overestimate shear stress. The curve 610 shows the strain histogram of one scaled shear stress study in comparison with the region 605 of manufacturing conditions for validated mixing protocols. Stated differently, region 605 represents the actual shear stress in validated mixing protocols of pharmaceutical products, while curve 610 represents the predicted shear stress of the scaled studies. The plot in FIG. 1 shows that the scaled studies have a higher shear stress than the typical operating range of mixing protocols.

The surrogate mixing studies, shear stress, and overmixing investigations associated with conventional mixing protocol development do not quantify air-liquid interfacial stress, air entrainment, and risk of visible or sub-visible particle formation. Therefore, these metrics are conventionally assessed with full-scale investigations using actual biopharmaceutical product. Full-scale investigations using product are expensive and time consuming. The cost and time constraints of full-scale investigations reduce repeatability and increase the difficulty of collecting enough samples to reduce sampling variability. Further, probes associated with the full-scale investigations may impact the flow associated with the mixing protocol, and provide inaccurate data. Because the nature of full-scale investigations are specific to the parameters of a given mixing protocol, the full-scale investigations require frequent re-validation.

Systems and methods disclosed herein may provide an improved development flow for mixing protocols. For example, the systems and methods described herein may allow for the development of predictive models that enable the high-throughput evaluation of mixing protocols. Predictive models may be generated that can quantify air-liquid interfacial stress, air entrainment, and risk of visible or sub-visible particle formation associated with a mixing protocol.

Referring to FIG. 2, a method 200 of developing a predictive model for evaluating mixing protocols may include mapping the design space 201, structuring a design of experiment (DOE) design 202, conducting a computational fluid dynamics (CFD) analysis 203, building candidate predictive models 204, and/or evaluating predictions 205.

Mapping the design space 201 may include identifying mixing protocol parameters that will be studied. Mixing protocol parameters may include “input variables” or aspects of the mixing protocol that may be adjusted, varied, controlled, and/or monitored to affect the outcome of the mixing protocol. Examples of mixing protocol parameters include, but are not limited to, impeller speed, batch size, solution viscosity, solution density, mixing vessel size, mixing vessel geometry, and mixing time.

Impeller speed may be quantified in terms of revolutions per minute (RPM) or as a percentage of maximum impeller speed. Batch size may refer to the volume of the mixing vessel load, as a percentage of the capacity of the mixing vessel. Solution viscosity and solution density are parameters specific to a protein of interest. During production, solution viscosity and density may be adjusted to achieve desired viscosity and density parameters prior to execution of a mixing protocol.

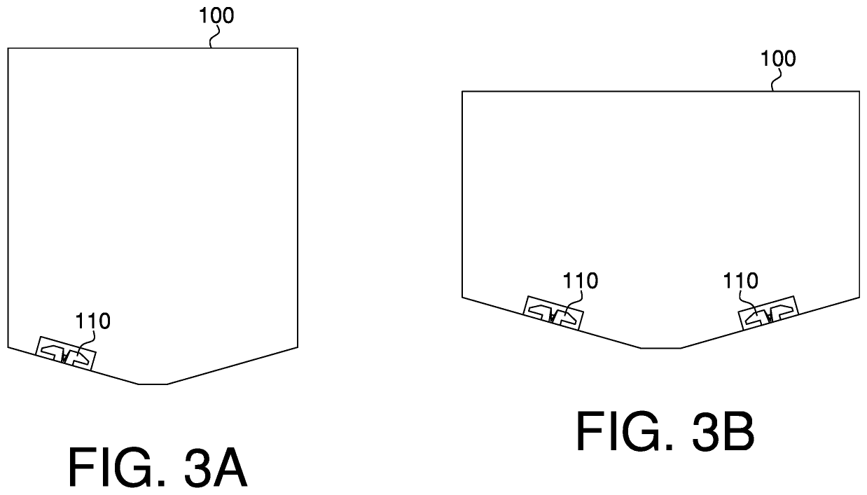

In additional to the potential mixing protocol parameters discussed above, mapping the design space may include identifying potential mixing vessel sizes and potential mixing vessel geometries. Mixing vessels may be provided with a variety of shapes and sizes. For example, mixing vessels may include cylindrical shapes, conical shapes, elliptical shapes, square shapes, or combinations thereof. Examples of mixing vessel geometries are shown in FIGS. 3A and 3B. The mixing vessel 100 shown in FIG. 3A includes a height and a width, where the height is greater than the width. The mixing vessel 100 shown in FIG. 3B includes a height and width, where the width is greater than the height. The proportion of height to width of mixing vessel 100 is a component of mixing vessel geometry and may affect the pattern of fluid flow within mixing vessel 100.

A mixing vessel 100 may include one or more mechanisms capable of providing agitation. For example, a mixing vessel 100 may include one or more impellers 110 that are capable of providing flow within the mixing vessel. The mixing vessel 100 shown in FIG. 3A includes one impeller 110, disposed on one side of mixing vessel 100. The mixing vessel 100 shown in FIG. 3B, includes two impellers 110, symmetrically disposed on opposing sides of mixing vessel 100. In addition, or alternatively, agitation within mixing vessel 100 may be provided by a concentrically mounted impeller, wave bags, a rocking actuator, or other means of agitating a solution within mixing vessel 100.

Although exemplary mixing vessel geometries are shown in FIGS. 3A and 3B, these are only two examples. In some embodiments, mixing vessel 100 may include baffles or other structures designed to alter fluid flow within mixing vessel 100. Mixing vessel geometries including other proportions, configurations, shapes, and mechanisms for providing agitation may be used with the systems and methods described herein.

In addition to identifying mixing protocol parameters, mapping the design space 201 may also include identifying evaluation criteria. Evaluation criteria may include “output variables” or aspects of the mixing protocol that depend on the values chosen for mixing protocol parameters. Examples of evaluation criteria include, but are not limited to, flow pattern, fluid velocity distribution, fluid flow vector field, fluid flow streamlines, blend time (e.g., steady state blend time or transient blend time), residence time distribution, contour shear strain rate, average shear strain rate, exposure analysis, power consumption, pressure, turbulent dissipation rate, and Kolmogorov length.

Referring to again to FIG. 2, a method 200 of developing a predictive model for evaluating mixing protocols may include structuring a DOE design 202. For example, a DOE design may be structured after mixing protocol parameters and evaluation criteria are identified. Design of Experiments (DOE) refers to a methodology of structuring experiments, simulations, and/or measurements that enables identification of multivariate interactions. As DOE is understood by those of ordinary skill in the art, it is not described in additional detail.

In the context of developing a predictive model for evaluating mixing protocols, structuring DOE design 202 includes selecting test values for each identified mixing protocol parameter and identifying the experiments, simulations, and measurements that will have to be performed in order to determine the evaluation criteria for each set of mixing protocol parameter test values.

For example, if impeller speed, batch size, solution viscosity, and mixing vessel size are identified as four mixing protocol parameters, structuring DOE design 202 includes selecting test values for impeller speed, batch size, solution viscosity, and mixing vessel size. In some embodiments, approximately 10 to approximately 500 test values, such as, for example, approximately 30 to approximately 100 test values may be selected for each mixing protocol parameter. Other numbers of test values may be selected for each mixing protocol parameter, such as, for example, less than approximately 10, or approximately 100 to approximately 1000. The accuracy of a subsequent CFD analysis is correlated to the amount of test values selected for each mixing protocol parameter, and more selecting more test values for some mixing protocol parameters may provide a more meaningful CFD analysis.

Referring to again to FIG. 2, a method 200 of developing a predictive model for evaluating mixing protocols may include conducting a computational fluid dynamics (CFD) analysis. For example, CFD analysis may be conducted after test values for each identified mixing protocol parameter, and the experiments, simulations, and measurements that will have to be performed in order to determine the evaluation criteria for each set of mixing protocol parameter test values are identified.

The CFD analysis may include one or more simulations indicative of fluid flow within mixing vessel 100. For example, CFD analysis may include a steady flow analysis, a transient flow analysis, a blend time analysis, and/or an exposure analysis. In particular, transient flow analysis may assist in evaluating acceleration time from rest to steady state velocity, evaluating the potential for frothing, foaming, or sloshing, and quantifying surface deformation (e.g., as part of an aggregate formation risk assessment).

The CFD analysis may be based on mathematical solutions to fluid flow models, including, but not limited to, laws of conservation, Naiver-Stokes equations, Euler equations, Bernoulli equations, compression wave equations, boundary layer equations, idealized flow, potential flow, duct flow, vortex formation, eddy formation, and turbulence formation. The CFD analysis may be performed by computer system running CFD analysis software, such as, for example, programs including the Star CCM, OpenFoam, Simulia, and Ansys Workbench systems.

In the context of the present disclosure, one or more mixing vessel geometries may be programmed into the computer system operating the analysis software in order to determine how mixing vessel geometry affects the evaluation criteria. For example, the dimensions and shape of mixing vessel 100, in addition to the size, shape, and placement of the mechanisms for causing agitation (e.g., impellers 110) may be modeled to frame the various flow simulations discussed above.

The results of the CFD analysis may include a vector map, a streamline map, a strain rate contour map, a strain histogram, a flow pattern, a fluid velocity distribution, a fluid flow vector field, fluid flow streamlines, a steady state blend time, a transient blend time, a residence time distribution, a contour shear strain rate, an average shear strain rate, an exposure analysis, a power consumption, a pressure, a turbulent dissipation rate, and/or a Kolmogorov length.

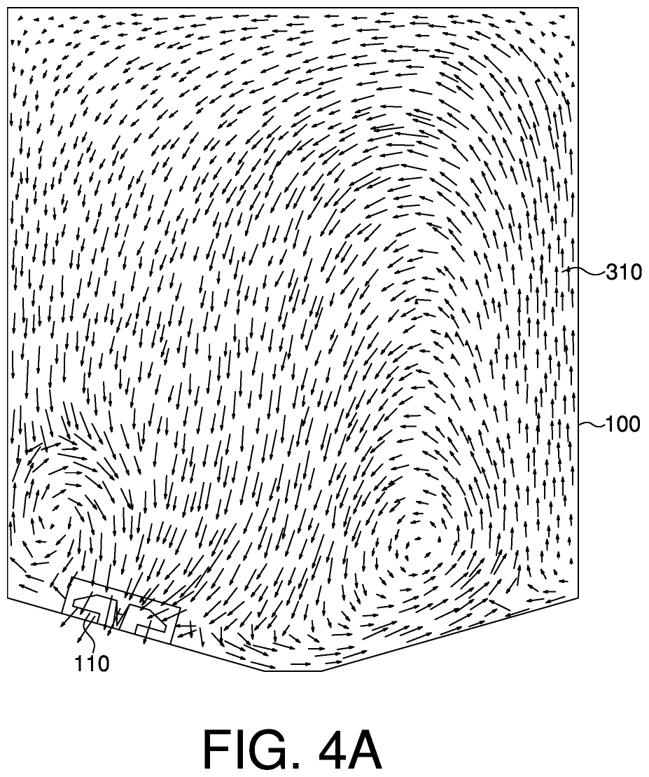

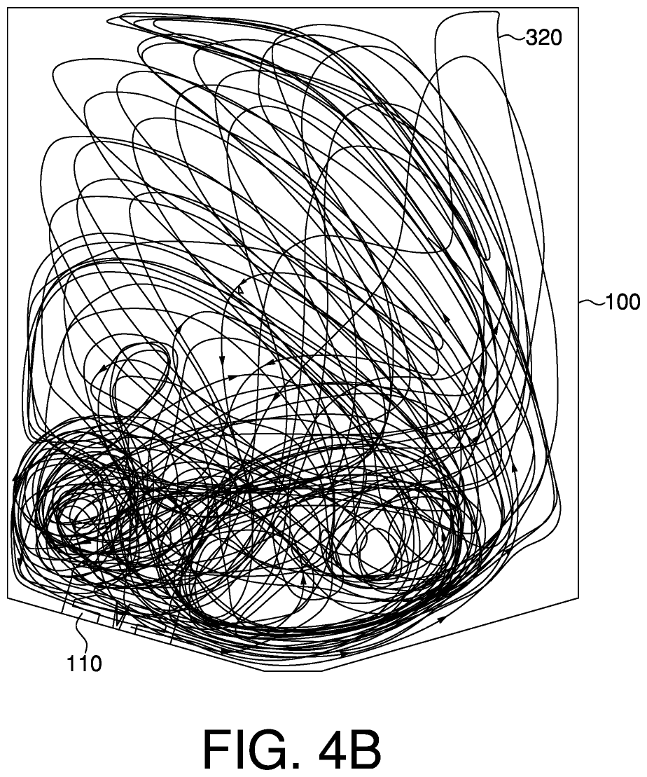

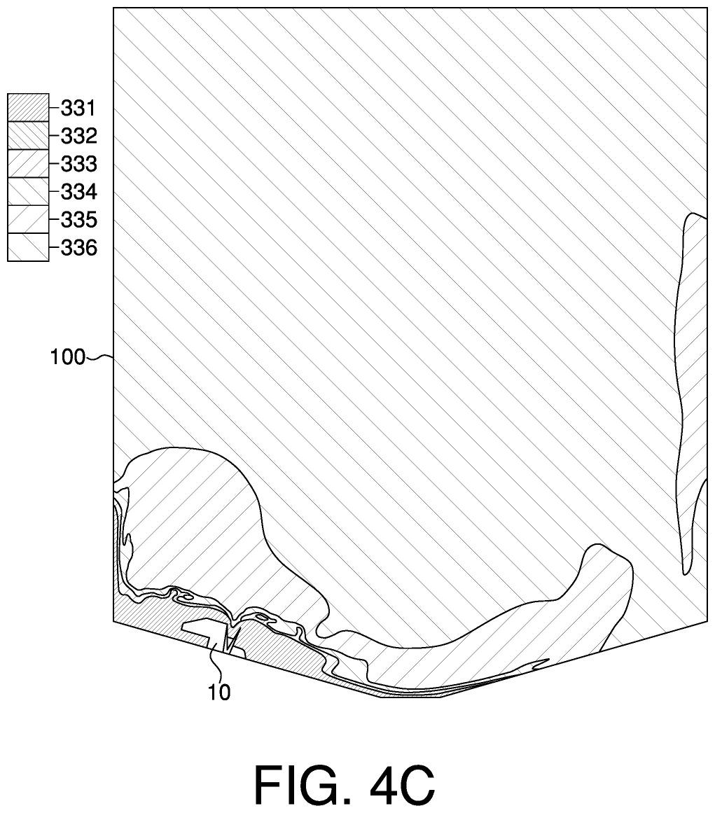

FIG. 4A shows an exemplary vector map generated as a result of CFD analysis. The vector map includes a plurality of vectors 310. The direction of each vector 310 indicates the direction of fluid flow at the location of the vector, and the magnitude of the vector indicates the velocity of fluid flow at the location of the vector. FIG. 4B shows a streamline map generated as a result of CFD analysis. The streamline map includes a plurality of streamlines 320. Each streamline represents a curve that is tangent to the velocity vector of the flow, and is indicative of where fluid elements will travel in a steady state.

Vector maps and streamline maps may be reviewed to determined stagnant areas, eddies, or other flow structures that could affect the efficacy of the mixing protocol. Vectors maps, streamline maps, or both, may be used to qualitatively compare different mixing protocol parameters (e.g., different mixing vessel geometries).

FIG. 4C shows a strain rate contour map depicted in black and white. In practice, different regions of the strain rate contour map may be represented by different colors. Table 1 shows approximate strain rates associated with labeled regions 331-336, and exemplary colors that may be used to represent the bins of strain rates shown in FIG. 4C and Table 1.

TABLE 1
Range of Strain Rates Associated with Regions of FIG. 4C

| Region Number | Strain Rate Range (s⁻¹) | Exemplary Color |
|---|---|---|
| 331 | 18.0-19.1 | Red |
| 332 | 15.8-17.9 | Orange |
| 333 | 13.2-15.7 | Yellow |
| 334 | 5.5-13.1 | Green |
| 335 | 2.5-5.4 | Light Blue |
| 336 | 0-2.4 | Dark Blue |

The bins of strain rates shown in Table 1 are one example. The grouping and distributions of strain rates may be altered depending on the range of strain rates observed during the CFD analysis. Evaluation criteria such as a strain histogram, average strain rate, and peak strain may be determined from the strain rate contour map. The strain rate contour map may be analyzed to determine regions of the mixing vessel that are subject to high levels of strain.

As previously described, criteria for quantitative and qualitative evaluation of mixing protocols (e.g., evaluation criteria) may be determined by CFD analysis. Evaluation criteria determined by CFD analysis correspond to a set of test values for the mixing protocol parameters. Relationships of evaluation criteria and the corresponding mixing protocol parameters may be used to assess the effect of variations in mixing protocol parameters on the overall utility and/or efficacy of a mixing protocol.

Referring again to FIG. 2, a method 200 of developing a predictive model for evaluating mixing protocols may include building candidate predictive models 204. For example, candidate predictive models may be built after evaluation criteria are determined for corresponding test values of mixing protocol parameters. For each identified evaluation criterion, one or more candidate predictive models may be chosen.

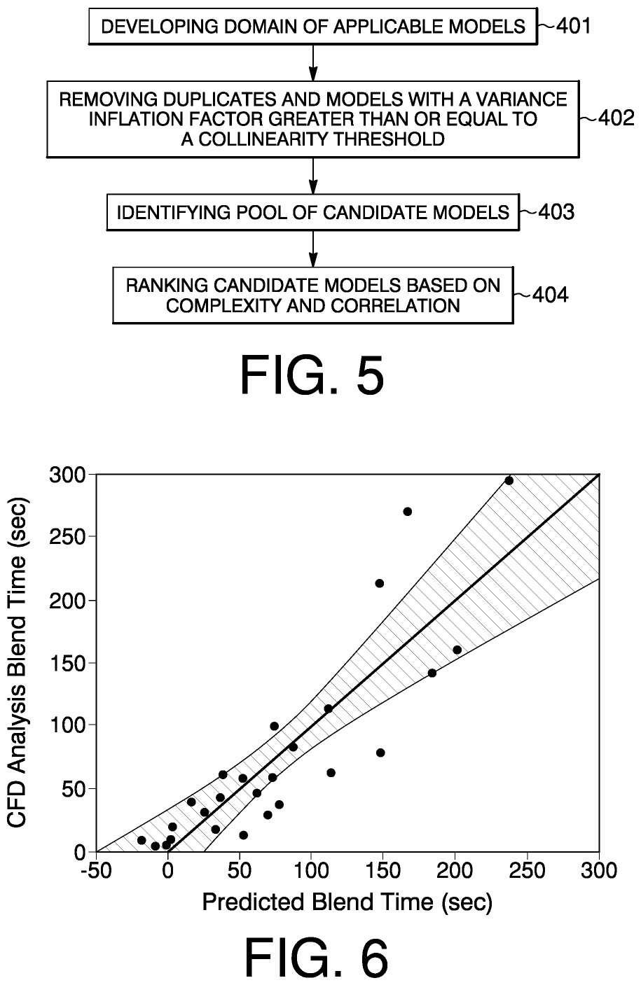

FIG. 5 shows an exemplary method of developing and ranking potential predictive models for an evaluation criterion. The method of developing and ranking potential predictive models may include developing a domain of applicable models (step 401), removing duplicate models and models with a variance inflation factor greater than or equal to a collinearity threshold (step 402), identifying a pool of candidate models (step 403), and ranking the candidate models based on complexity and correlation (step 404). The method of developing and ranking potential predictive models may be applied to each of the evaluation criteria identified in the DOE design in order to generate a ranked pool of candidate predictive models for each evaluation criterion.

Developing and ranking potential predictive models includes developing a domain of applicable models for a given evaluation criterion based on the identified mixing protocol parameters. In this context, model refers to an algebraic expression relating the evaluation criterion to the mixing protocol parameters. Single variate, bivariate, trivariate, and other multivariate interactions between mixing protocol parameters are considered when developing the domain of applicable models. For example, products, quotients, exponents, and other multivariate relationships of mixing protocol parameters may be considered, The domain of applicable models may also include known mechanistic or empirical relationships. In some embodiments, the domain of applicable models includes tens of thousands of models, such as, for example, greater than 50,000 potential predictive models.

After the domain of models are developed, models with duplicate parameters may be removed. For example, in developing algebraic expressions relating evaluation criterion to mixing protocol parameters, equivalent expressions may be created. These equivalent expressions may functionally be duplicates that can be removed from the domain. The variance inflation factor (VIF) of each remaining model may be calculated and models with a VIF greater than or equal to a collinearity threshold may be removed from the domain. In some embodiments, the collinearity threshold is four or less, such as, for example, two, three, or four. After models with duplicate parameters and models with a VIF greater than or equal to a collinearity threshold are removed, the remaining subset of models may include hundreds of models. For example, the remaining subset of models may include less than or equal to 500 models.

A pool of candidate predictive models may be identified from the remaining subset of models. For example, a pool of candidate predictive models may include univariate models, bivariate models and trivariate models. Each candidate predictive model from the pool of candidate predictive models may have a R² value of greater than or equal to approximately 0.70. In some embodiments, each candidate predictive model from the pool of candidate predictive models may have a R² value can be greater than or equal to approximately the following values: 0.60, 0.70, 0.75, 0.80, 0.85, 0.9, or 0.95. In some embodiments, the pool of candidate predictive models includes the univariate model with the greatest R² value, the bivariate model with the greatest R² value, and the trivariate model with the greatest R² value. After the pool of candidate predictive models are identified, the candidate predictive models may be ranked according to complexity and correlation. For example, predictive models that have higher correlation to data obtained from CFD analysis (e.g., a higher R² value) may be higher ranked according to correlation, while predictive models that have less complexity (e.g., fewer terms) may have a more positive complexity ranking. Further examples of evaluating the correlation of potential predictive models to the results of CFD analysis are described in the Examples section below. These two rankings may be combined such that predictive models that have higher correlation to data obtained from CFD analysis (e.g., a higher R² value) with the least complexity (e.g., fewer terms) may be higher ranked than predictive models that have lower R² values and/or more complexity.

After the predictive models are ranked according to complexity and correlation, a predictive model may be selected according to desired complexity and correlation attributes. The selected predictive model may be further studied for other mixing protocol parameters test values or types of mixing protocols.

Referring again to FIG. 2, a method 200 of developing a predictive model for evaluating mixing protocols may include evaluating predictions. Candidate predictive models may be tested over an increased range of mixing protocol parameter test values to generate predicted evaluation criteria.

The predicted evaluation criteria may be compared to full-scale investigations or CFD analysis to validate the selected predictive model. Once a predictive model is validated for an evaluation criterion and a set of mixing protocol parameters, the model can be used to evaluate thousands of mixing protocols in a high-throughput manner. The rate at which mixing protocols can be evaluated with the predictive model may enable solving for optimum mixing protocol parameter conditions.

In addition, or alternatively, known mechanistic or empirical relationships from the literature may be compared to the candidate predictive models. If the correlation of the candidate predictive models is improved by the addition of a term from known or empirical relationship, the term may be incorporated into the candidate predictive model.

As more predictive models are generated and the library of CFD analysis data grows, more accurate predictive models will be generated for each identified evaluation criteria. All evaluation criteria for all mixing protocol parameters may be assessed in the aforementioned high-throughput manner, to determine which mixing protocol parameters result in sufficient evaluation criteria.

Advantageously, the high-throughput manner of evaluating mixing protocols may result in significant time savings in identifying mixing protocols suitable for use in the production of biopharmaceutical products.

### EXAMPLES

### Example 1

A CFD blend time analysis was conducted with blend time as an evaluation criterion, and batch size, impeller speed, and solution viscosity identified as mixing protocol parameters. A candidate predictive model was identified, and is described by Equation 1:

T_{blend}=c₁+c₂X₁+c₃X₃+c₄X₂X₃ Eq. (1)

where T_{blend} is the blend time, X₁ is the batch size, X₂ is the impeller speed, X₃ is the solution viscosity, and c₁, c₂, c₃, and c₄ are constants. The T_{blend} determined by CFD for the test values of the mixing protocol parameters is plotted versus the T_{blend} determined by Equation 1, and is shown in FIG. 6. A line of 1:1 correlation and a region around the line of 1:1 correlation are also shown in FIG. 6 to illustrate the correlation of the predictive model to the results of CFD analysis. The predictive model was found to have better correlation to the CFD analysis than known relationships from Flickinger and Nienow, Scale-Up, Stirred Tank Reactors, Encyclopedia of Industrial Biotechnology (2010).

### Example 2

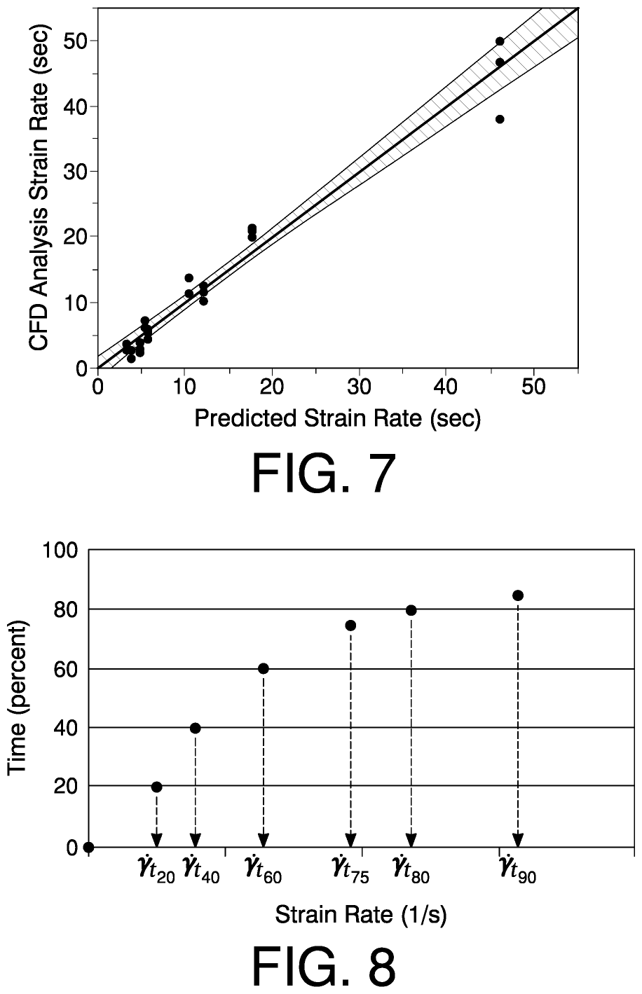

A CFD strain contour analysis was conducted with average strain rate as an evaluation criterion, and batch size, impeller speed, and solution viscosity identified as mixing protocol parameters. A candidate predictive model was identified, and is described by Equation 2:

$${γ}_{mean} = {c}_{1} + {c}_{2} {X}_{2} + {c}_{3} \sqrt{\frac{{X}_{2}^{3}}{{X}_{1}}} \qquad Eq . ( 2 )$$

where γₘₑₐₙ is the average strain rate, X₁ is the batch size, X₂ is the impeller speed, and c₁, c₂, and c₃, are constants. The γₘₑₐₙ determined by CFD for the test values of the mixing protocol parameters is plotted versus the γₘₑₐₙ determined by Equation 2, and is shown in FIG. 7. A line of 1:1 correlation and a region around the line of 1:1 correlation are also shown in FIG. 7 to illustrate the correlation of the predictive model to the results of CFD analysis. The correlation of the predictive model was improved by the addition of the

$${c}_{3} \sqrt{\frac{{X}_{2}^{3}}{{X}_{1}}}$$

term, based on the relationships described in Ladner et al., CFD Supported Investigation of Shear Induced by Bottom-Mounted Magnetic Stirrer in Monoclonal Antibody Formulation, Pharm. Res. 35(11): 215, Sep. 25, 2018.

### Example 3

Strain rate histograms may be generated by CFD analysis. However, the generation of a strain rate histogram for one combination of test values is time intensive. A more efficient methodology may include generating a predictive model to describe cumulative strain and plotting a strain rate histogram based on the predictive model. An example of a strain rate histogram generated using a predictive model is shown in FIG. 8.

Referring to FIG. 8, points for a strain rate histogram (e.g., points for t=20, t=40, t=60, t=75, t=80, and t=90) were generated according to a predictive model for strain rate (Equation 2). The points were plotted in a histogram, shown in FIG. 8. The cumulative strain described by the histogram can be generated faster, and with less associated labor, than a traditional CFD-based exposure analysis.

### Example 4

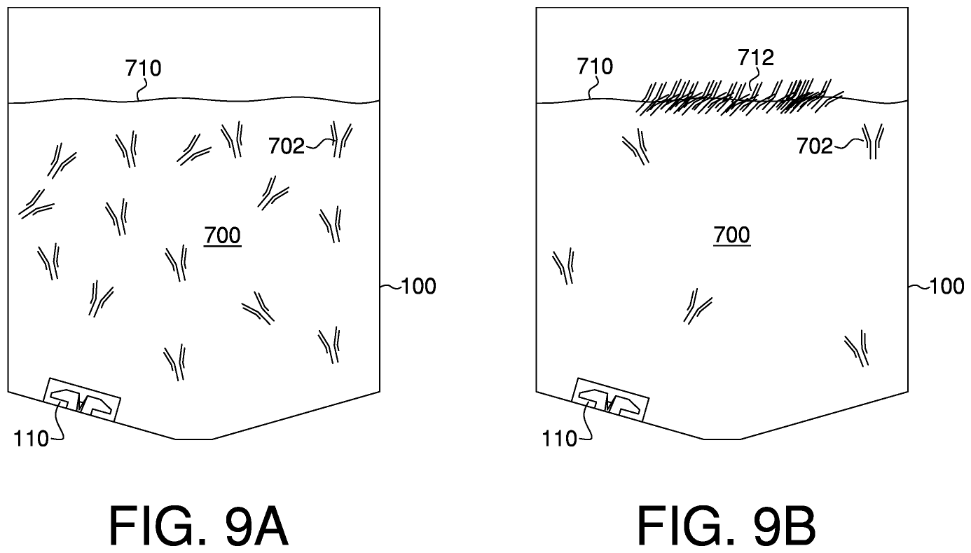

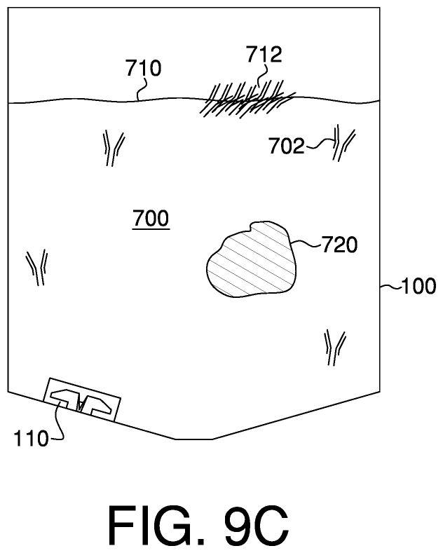

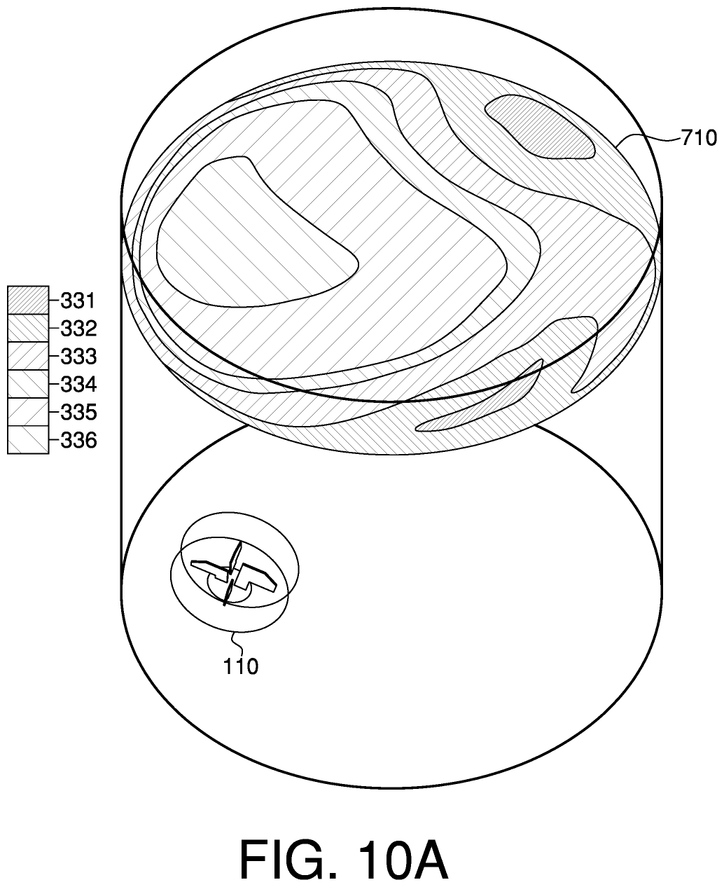

Without being limited by theory, a possible mechanism of visible and sub-visible particle formation is shown in FIGS. 9A-9C. Individual proteins 702 (e.g., host cell protein, proteins of interest, etc.) may be present in solution 700 within mixing vessel 100. As shown in FIG. 9A, the surface 710 of solution 700 may initially be clear of protein aggregates 712.

Proteins 702 may deform in response to surface tension upon adsorption at the air-liquid interface (e.g., at surface 710). Upon deformation, charged regions of proteins 702 may be exposed. The exposed charge regions may aggregate due to the thermodynamic environment. The aggregated proteins may form a network 712 at the surface 710, shown in FIG. 9B. When the surface 710 of solution 700 is disturbed (e.g., due to a mixing protocol) the network 712 may be broken and pieces of the broken network 712 may be drawn into the bulk of solution 700.

The pieces of the broken network may aggregate with other proteins 702, to form a larger network 712, which will again break up and be drawn into the bulk of the solution 700. When the pieces of a protein network reach a sufficient size, they are detected as large aggregates 720 within solution 700, shown in FIG. 9C. The large aggregates 720 may appear as visible particles and may cloud solution 700.

Conventional means of addressing risk of particle formation rely on the study of hydrodynamic shear. However, hydrodynamic shear does not account for protein aggregate formation, and shear-based scaled tests do not predict all production-scale evaluation criteria. Risk of particle formation continues to be an obstacle to mixing protocol development due to difficulty in quantifying the impact of particle formation, variation in filter performance, and lack of understanding of the long-term behavior of visible and sub-visible particles in solution.

Stress at the air-liquid interface is most likely the dominant factor in aggregate formation. Air entrainment also contributes to aggregate formation. The involvement of air entrainment in aggregate formation is supported by surface tension and free energy estimates, as well as atomic-force microscopy observation. Solid-liquid interfacial stresses, cavitation, nucleation, and thermal stress may contribute secondarily to the formation of aggregates.

In order to better quantify risk of particle formation, a predictive model may be developed according to embodiments described herein that describes risk of particle formation as a function of mixing protocol parameters. Possible mixing protocol parameters include characteristics of the aggregating proteins, excipient profile of the solution, and environmental factors (e.g., temperature, pressure, etc.).

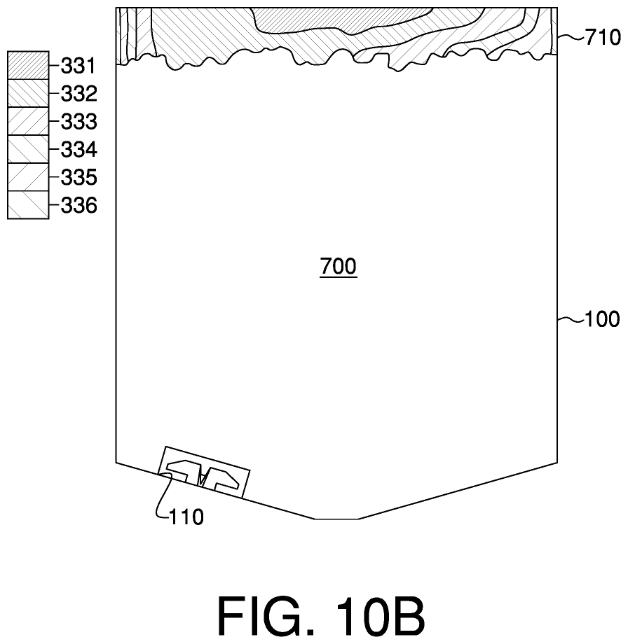

A CFD analysis can determine vertical velocity contours and volume-averaged velocity. FIG. 10A shows an example of vertical velocity contours, determined by CFD. FIG. 10B shows an example of volume-averaged velocity, determined by CFD. In this context, volume-averaged velocity refers to a spatially averaged fluid velocity in a volume near the surface of the liquid.

Similar to the strain rate contour map described previously (FIG. 4C), in practice, different regions of the vertical velocity contour map and volume-averaged velocity may be represented by different colors, assigned according to the velocity of the region. Table 2 shows exemplary colors that may be used to represent the regions of varying vertical velocity shown in FIG. 10A and regions of varying volume-averaged vertical velocity shown in FIG. 10B.

TABLE 2
Exemplary Colors Associated with Regions of FIGS. 10A and 10B

| Region Number | Exemplary Color |
|---|---|
| 331 | Red |
| 332 | Orange |
| 333 | Yellow |
| 334 | Green |
| 335 | Light Blue |
| 336 | Dark Blue |

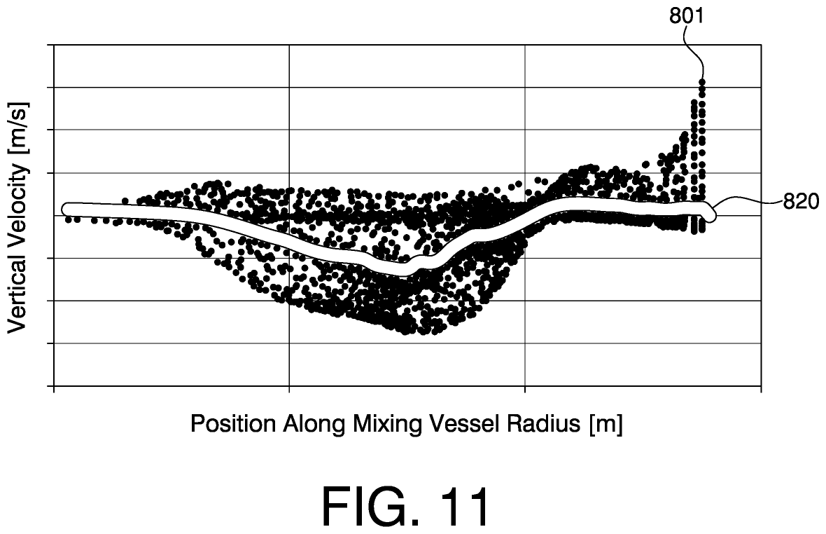

The vertical velocity contours determined by CFD may be plotted versus their location in the mixing vessel in order to illustrate the relationship between relative differences in vertical velocity as a function of position. For example, the CFD analysis may determine a vertical velocity value for each computational cell of the mixing vessel. That vertical velocity may be plotted versus the linear displacement along a radius from the center of the mixing vessel, as shown in FIG. 11. Each measurement 801 corresponds to a computational cell with vertical velocity and a position along a radius of the mixing vessel. A weighted average 820 may be determined from the individual measurements 801, where the weight assigned to each measurement 801 correlates to the volume of the computational cell corresponding to that measurement 801.

A predictive model for assessing the risk of aggregate formation may be developed using the aforementioned techniques. For example, a relationship between characteristics of the aggregating proteins, excipient profile of the solution, environmental factors, and aggregate formation may be determined using vertical velocity contours or volume-averaged velocity determined by CFD.

The present disclosure is further described by the following non-limiting items.

Item 1. A method of developing a predictive model, the method comprising:

a) identifying mixing protocol parameters for the predictive model;

b) selecting test values for the mixing protocol parameters;

c) conducting a computational fluid dynamics (CFD) simulation for each combination of test values;

d) generating a domain of potential predictive models relating to the mixing protocol parameters; and ranking the domain of potential predictive models relating to the mixing protocol parameters.

Item 2. The method of item 1, further comprising:

identifying an evaluation criterion for the predictive model after step (a); identifying a CFD simulation required to be performed in order to generate the evaluation criterion after step (b);

identifying a pool of candidate predictive models from the domain of potential predictive models after step (d); and

ranking the pool of candidate predictive models.

Item 3. The method of any one of items 1 or 2, wherein the mixing protocol parameters include two or more of: impeller speed, batch size, solution viscosity, solution density, mixing vessel size, and mixing vessel geometry.

Item 4. The method of item 1, wherein the evaluation criteria include two or more of: flow pattern, fluid velocity distribution, fluid flow vector field, fluid flow streamlines, steady state blend time, transient blend time, residence time distribution, contour shear strain rate, average shear strain rate, exposure analysis, and power consumption.

Item 5. The method of any one of items 2 or 4, wherein the identified CFD simulation includes a steady flow analysis, a transient flow analysis, a blend time analysis, and/or an exposure analysis.

Item 6. The method of item 2, further comprising, after generating a domain of potential predictive models, and prior to identifying a pool of candidate predictive models:

calculating a variance inflation factor for each potential predictive model in the domain of potential predictive models; and

removing potential predictive models from the domain of potential predictive models that have a variance inflation factor greater than or equal to a collinearity threshold, thereby generating a subset of potential predictive models.

Item 7. The method of item 6, wherein the pool of candidate predictive models includes a univariate model from the subset that has a R² value higher than all other univariate models in the subset, and a bivariate model from the subset that has a R² value higher than all other bivariate models in the subset.

Item 8. The method of item 2, wherein ranking the pool of candidate predictive models includes ranking the pool of candidate predictive models based on number of terms, ranking the pool of candidate predictive models based on R² value, or both.

Item 9. The method of any one of items 1 to 8, wherein the test values are first test values, and the method further comprises:

using a candidate predictive model from the pool of candidate predictive models, generating an estimated value of the evaluation criteria corresponding to a combination of second test values.

Item 10. The method of item 9, wherein the method further comprises:

conducting the CFD simulation for the combination of second test values to generate an evaluation criterion corresponding to the combination of second test values; and

comparing the evaluation criterion corresponding to the combination of second test values with the estimated value of the evaluation criterion corresponding to the combination of second test values.

Item 11. A method of developing predictive models, the method comprising:

identifying first, second, and third mixing protocol parameters for the predictive models;

identifying first and second evaluation criteria for the predictive models;

selecting first test values for the first mixing protocol parameter;

selecting second test values for the second mixing protocol parameter;

selecting third test values for the third mixing protocol parameter;

identifying a first computational fluid dynamics (CFD) simulation required to be performed in order to generate the first evaluation criterion;

identifying a second CFD simulation required to be performed in order to generate the second evaluation criterion;

generating a first evaluation criterion corresponding to each combination of first test values, second values, and third test values, by performing the first CFD simulation for each combination of first test values, second values, and third test values;

generating a second evaluation criterion corresponding to each combination of first test values, second values, and third test values, by performing the second CFD simulation for each combination of first test values, second values, and third test values;

generating a first domain of first predictive models relating the first, second, and third mixing protocol parameters to the first evaluation criterion; and

generating a second domain of second predictive models relating the first, second, and third mixing protocol parameters to the second evaluation criterion.

Item 12. The method of item 11, further comprising:

calculating a variance inflation factor for each first predictive model and each second predictive model;

removing first predictive models from the first domain of first predictive models that have a variance inflation factor greater than or equal to three, thereby generating a first subset of first predictive models;

removing second predictive models from the second domain of second predictive models that have a variance inflation factor greater than or equal to three, thereby generating a first subset of first predictive models;

identifying a first pool of candidate first predictive models comprising a univariate model from the first subset that has a R² value higher than all other univariate models in the first subset, a bivariate model from the first subset that has a R² value higher than all other bivariate models in the first subset, and a trivariate model from the first subset that has a R² value higher than all other trivariate models in the first subset; and

identifying a second pool of candidate second predictive models comprising a univariate model from the second subset that has a R² value higher than all other univariate models in the second subset, a bivariate model from the second subset that has a R² value higher than all other bivariate models in the second subset, and a trivariate model from the second subset that has a R² value higher than all other trivariate models in the second subset.

Item 13. The method of item 12, further comprising:

selecting fourth test values for the first mixing protocol parameter;

selecting fifth test values for the second mixing protocol parameter;

selecting sixth test values for the third mixing protocol parameter;

generating an estimated first evaluation criterion corresponding to each combination of fourth test values, fifth test values, and sixth test values, using each candidate first predictive model of the first pool of candidate first predictive models;

generating a first evaluation criterion corresponding to each combination of fourth test values, fifth test values, and sixth test values, by performing the first CFD simulation for each combination of fourth test values, fifth test values, and sixth test values; and

comparing the estimated first evaluation criterions generated by each candidate first predictive model of the first pool of candidate first predictive models to the first evaluation criterions corresponding to each combination of fourth test values, fifth test values, and sixth test values.

Item 14. The method of item 13, further comprising:

generating an estimated second evaluation criterion corresponding to each combination of fourth test values, fifth test values, and sixth test values, using each candidate second predictive model of the second pool of candidate second predictive models;

generating a second evaluation criterion corresponding to each combination of fourth test values, fifth test values, and sixth test values, by performing the second CFD simulation for each combination of fourth test values, fifth test values, and sixth test values; and

comparing the estimated second evaluation criterions generated by each candidate second predictive model of the second pool of candidate second predictive models to the second evaluation criterions corresponding to each combination of fourth test values, fifth test values, and sixth test values.

Item 15. The method of item 14, further comprising:

selecting a first predictive model from the first pool of candidate first predictive models, based on the comparison the estimated first evaluation criterions to the first evaluation criterions corresponding to each combination of fourth test values, fifth test values, and sixth test values;

selecting a second predictive model from the second pool of candidate second predictive models, based on the comparison the estimated first evaluation criterions to the first evaluation criterions corresponding to each combination of fourth test values, fifth test values, and sixth test values;

using the first predictive model, determining a first evaluation criterion corresponding to a mixing protocol; and

using the second predictive model, determining a second evaluation criterion corresponding to the mixing protocol.

Item 16. The method of item 9, wherein the first and second evaluation criteria are selected from a list comprising: flow pattern, fluid velocity distribution, fluid flow vector field, fluid flow streamlines, steady state blend time, transient blend time, residence time distribution, contour shear strain rate, average shear strain rate, exposure analysis, and power consumption.

Item 17. A method of modeling shear strain associated with a mixing protocol, the method comprising:

identifying mixing protocol parameters for a predictive model;

selecting test values for the mixing protocol parameters;

conducting a computational fluid dynamics exposure analysis for each of combination of test values, thereby generating a shear strain corresponding to each combination of test values;

identifying a pool of candidate predictive models;

ranking the pool of candidate predictive models;

selecting a predictive model from the pool of candidate predictive models; and

using the predictive model, evaluating cumulative shear strain of the mixing protocol at a plurality of time intervals to generate shear strain histogram data.

Item 18. The method of item 17, wherein the mixing protocol parameters include two or more of: impeller speed, batch size, solution viscosity, solution density, mixing vessel size, and mixing vessel geometry.

Item 19. The method of item 17, wherein the mixing protocol is a mixing protocol associated with biopharmaceutical products in a bioreactor.

Item 20. The method of item 17, further comprising using the shear strain histogram data to assess the risk of visible or sub-visible particle formation.

Item 21. The method of item 17, wherein ranking the pool of candidate predictive models includes ranking the pool of candidate predictive models based on number of terms, ranking the pool of candidate predictive models based on R² value, or both; and

selecting a predictive model from the pool of candidate predictive models includes selecting the model with the highest R² value.

Those skilled in the art will appreciate that the conception upon which this disclosure is based may readily be used as a basis for designing other methods and systems for carrying out the several purposes of the present disclosure. Accordingly, the claims are not to be considered as limited by the foregoing description.

1. A method of developing a predictive model, the method comprising:
a) identifying mixing protocol parameters for the predictive model;
b) selecting test values for the mixing protocol parameters;
c) conducting a computational fluid dynamics (CFD) simulation for each combination of test values;
d) generating a domain of potential predictive models relating to the mixing protocol parameters; and
e) ranking the domain of potential predictive models relating to the mixing protocol parameters.

2. The method of claim 1, further comprising:
identifying an evaluation criterion for the predictive model after step (a);
identifying a CFD simulation required to be performed in order to generate the evaluation criterion after step (b);
identifying a pool of candidate predictive models from the domain of potential predictive models after step (d); and
ranking the pool of candidate predictive models.

3. The method of claim 1, wherein the mixing protocol parameters include two or more of: impeller speed, batch size, solution viscosity, solution density, mixing vessel size, and mixing vessel geometry.

4. The method of claim 2, wherein the evaluation criterion includes two or more of: flow pattern, fluid velocity distribution, fluid flow vector field, fluid flow streamlines, steady state blend time, transient blend time, residence time distribution, contour shear strain rate, average shear strain rate, exposure analysis, and power consumption.

5. The method of claim 2, wherein the identified CFD simulation includes a steady flow analysis, a transient flow analysis, a blend time analysis, and/or an exposure analysis.

6. The method of claim 2, further comprising, after generating a domain of potential predictive models, and prior to identifying a pool of candidate predictive models:
calculating a variance inflation factor for each potential predictive model in the domain of potential predictive models; and
removing potential predictive models from the domain of potential predictive models that have a variance inflation factor greater than or equal to a collinearity threshold, thereby generating a subset of potential predictive models.

7. The method of claim 6, wherein the pool of candidate predictive models includes a univariate model from the subset that has a R² value higher than all other univariate models in the subset, and a bivariate model from the subset that has a R² value higher than all other bivariate models in the subset.

8. The method of claim 2, wherein ranking the pool of candidate predictive models includes ranking the pool of candidate predictive models based on number of terms, ranking the pool of candidate predictive models based on R² value, or both.

9. The method of claim 2, wherein the test values are first test values, and the method further comprises:
using a candidate predictive model from the pool of candidate predictive models, generating an estimated value of the evaluation criteria corresponding to a combination of second test values.

10. The method of claim 9, wherein the method further comprises:
conducting the CFD simulation for the combination of second test values to generate an evaluation criterion corresponding to the combination of second test values; and
comparing the evaluation criterion corresponding to the combination of second test values with the estimated value of the evaluation criterion corresponding to the combination of second test values.

11. A method of developing predictive models, the method comprising:
identifying first, second, and third mixing protocol parameters for the predictive models;
identifying first and second evaluation criteria for the predictive models;
selecting first test values for the first mixing protocol parameter;
selecting second test values for the second mixing protocol parameter;
selecting third test values for the third mixing protocol parameter;
identifying a first computational fluid dynamics (CFD) simulation required to be performed in order to generate the first evaluation criterion;
identifying a second CFD simulation required to be performed in order to generate the second evaluation criterion;
generating a first evaluation criterion corresponding to each combination of first test values, second test values, and third test values, by performing the first CFD simulation for each combination of first test values, second test values, and third test values;
generating a second evaluation criterion corresponding to each combination of first test values, second test values, and third test values, by performing the second CFD simulation for each combination of first test values, second test values, and third test values;
generating a first domain of first predictive models relating the first, second, and third mixing protocol parameters to the first evaluation criterion; and
generating a second domain of second predictive models relating the first, second, and third mixing protocol parameters to the second evaluation criterion.

12. The method of claim 11, further comprising:
calculating a variance inflation factor for each first predictive model and each second predictive model;
removing first predictive models from the first domain of first predictive models that have a variance inflation factor greater than or equal to three, thereby generating a first subset of first predictive models;
removing second predictive models from the second domain of second predictive models that have a variance inflation factor greater than or equal to three, thereby generating a second subset of first predictive models;
identifying a first pool of candidate first predictive models comprising a univariate model from the first subset that has a R² value higher than all other univariate models in the first subset, a bivariate model from the first subset that has a R² value higher than all other bivariate models in the first subset, and a trivariate model from the first subset that has a R² value higher than all other trivariate models in the first subset; and
identifying a second pool of candidate second predictive models comprising a univariate model from the second subset that has a R² value higher than all other univariate models in the second subset, a bivariate model from the second subset that has a R² value higher than all other bivariate models in the second subset, and a trivariate model from the second subset that has a R² value higher than all other trivariate models in the second subset.

13. The method of claim 12, further comprising:
selecting fourth test values for the first mixing protocol parameter;
selecting fifth test values for the second mixing protocol parameter;
selecting sixth test values for the third mixing protocol parameter;
generating an estimated first evaluation criterion corresponding to each combination of fourth test values, fifth test values, and sixth test values, using each candidate first predictive model of the first pool of candidate first predictive models;
generating a first evaluation criterion corresponding to each combination of fourth test values, fifth test values, and sixth test values, by performing the first CFD simulation for each combination of fourth test values, fifth test values, and sixth test values; and
comparing the estimated first evaluation criterions generated by each candidate first predictive model of the first pool of candidate first predictive models to the first evaluation criterions corresponding to each combination of fourth test values, fifth test values, and sixth test values.

14. The method of claim 13, further comprising:
generating an estimated second evaluation criterion corresponding to each combination of fourth test values, fifth test values, and sixth test values, using each candidate second predictive model of the second pool of candidate second predictive models;
generating a second evaluation criterion corresponding to each combination of fourth test values, fifth test values, and sixth test values, by performing the second CFD simulation for each combination of fourth test values, fifth test values, and sixth test values; and
comparing the estimated second evaluation criterions generated by each candidate second predictive model of the second pool of candidate second predictive models to the second evaluation criterions corresponding to each combination of fourth test values, fifth test values, and sixth test values.

15. The method of claim 14, further comprising:
selecting a first predictive model from the first pool of candidate first predictive models, based on the comparison the estimated first evaluation criterions to the first evaluation criterions corresponding to each combination of fourth test values, fifth test values, and sixth test values;
selecting a second predictive model from the second pool of candidate second predictive models, based on the comparison the estimated first evaluation criterions to the first evaluation criterions corresponding to each combination of fourth test values, fifth test values, and sixth test values;
using the first predictive model, determining a first evaluation criterion corresponding to a mixing protocol; and
using the second predictive model, determining a second evaluation criterion corresponding to the mixing protocol.

16. The method of claim 9, wherein the first and second evaluation criteria are selected from a list comprising: flow pattern, fluid velocity distribution, fluid flow vector field, fluid flow streamlines, steady state blend time, transient blend time, residence time distribution, contour shear strain rate, average shear strain rate, exposure analysis, and power consumption.

17. A method of modeling shear strain associated with a mixing protocol, the method comprising:
identifying mixing protocol parameters for a predictive model;
selecting test values for the mixing protocol parameters;
conducting a computational fluid dynamics exposure analysis for each of combination of test values, thereby generating a shear strain corresponding to each combination of test values;
identifying a pool of candidate predictive models;
ranking the pool of candidate predictive models;
selecting a predictive model from the pool of candidate predictive models; and
using the predictive model, evaluating cumulative shear strain of the mixing protocol at a plurality of time intervals to generate shear strain histogram data.

18. The method of claim 17, wherein the mixing protocol parameters include two or more of: impeller speed, batch size, solution viscosity, solution density, mixing vessel size, and mixing vessel geometry.

19. The method of claim 17, wherein the mixing protocol is a mixing protocol associated with biopharmaceutical products in a bioreactor.

20. The method of claim 17, further comprising using the shear strain histogram data to assess the risk of visible or sub-visible particle formation.

21. The method of claim 17, wherein ranking the pool of candidate predictive models includes ranking the pool of candidate predictive models based on number of terms, ranking the pool of candidate predictive models based on R² value, or both; and
selecting a predictive model from the pool of candidate predictive models includes selecting the model with the highest R² value.
